(19) United States
(12) Patent Application Publication
(10) Pub. No.: US 2006/0062748 A1
(43) Pub. Date: Mar. 23, 2006

(54) Cosmetic composition comprising at least one cage-like structure compound

(76) Inventor: Estelle Mathonneau, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/222,734

(22) Filed: Sep. 12, 2005

Related U.S. Application Data
(60) Provisional application No. 60/620,670, filed on Oct. 22, 2004.

(30) Foreign Application Priority Data
Sep. 10, 2004 (FR) 04 52017

(51) Int. Cl. A61K 8/00

(52) U.S. Cl. 424/70.1

(57) ABSTRACT

The present disclosure relates to a cosmetic composition comprising, in a cosmetically acceptable medium, at least one compound having an opened or a closed cage-like structure, wherein the at least one compound comprises at least 4 atoms of an element chosen from columns 3 to 13 of the Periodic Table, and at least 4 oxygen atoms, wherein the atoms of the element are linked to oxygen atoms and at least one substituent, which are the same or different.

This application claims benefit of U.S. Provisional Application No. 60/620,670, filed Oct. 22, 2004, the contents of which are incorporated herein by reference. This application also claims benefit of priority under 35 U.S.C. § 119 to French Patent Application No. 04 52017, filed Sep. 10, 2004, the contents of which are also incorporated by reference.

The present disclosure relates to a cosmetic composition comprising, in a cosmetically acceptable medium, at least one compound having a cage-like structure comprising atoms of an element chosen from columns 3 to 13 of the Periodic Table (MERCK INDEX, 12th edition), as well as a method for hair shaping. The present disclosure also relates to the use of a compound having a cage-like structure to improve the lifting effect of skin care cosmetic products, to improve the resistance of the polymers used in nail varnish polishes, and to improve the lash curving effect in mascara products.

Hair styling products used to non permanently fix the hair may typically come as fixing polymer containing gels, solutions or sprays.

Nevertheless, these products may only make it possible to fix the hair within a time limit, even with an increased amount of fixing polymer. Moreover, these products may create a hair tacky feel.

Perm products may enable a long-lasting hair shaping.

Typically, the usual practice to obtain a permanent reshaping of the hair comprises, in a first step, opening the keratin disulfide bonds (cystine) of the hair by applying onto the hair, which has been straightened or placed beforehand under tension with suitable means (e.g., rollers or equivalent), a reducing composition (i.e., reducing step), then optionally after rinsing the treated hair, reforming in a second step the disulfide bonds by applying onto the hair under tension an oxidizing composition (i.e., oxidizing step, also called fixing step) to give lastly the desired form to the hair.

The new shape that is imposed to the hair by means of a chemical treatment such as herein, may be long lasting and for example, may stand the test of water or shampoo washing.

Nevertheless, such a method may not be fully satisfactory. While it is very efficient to reshape the hair, this type of reshaping may cause great damage to the hair fibers and for example, affects the hair feel.

Moreover, some cosmetic products applied onto the skin are expected to have a lifting effect. Polymers used in cosmetic compositions may have a relative resilience, but also may have a good mechanical resistance.

In fact, currently used polymers may typically have good resilience properties, but often suffer from poor mechanical ones.

United Kingdom Patent No. GB 1 556 376 uses a composition comprising a compound (O═Al—X)ₙ, wherein X is a carboxylic group, to form flexible films, which firmly adhere to the skin. Nevertheless, these aluminium-based compounds do not exhibit a cage-like structure.

The present inventor has surprisingly discovered that using cage-like structured compounds comprising atoms of an element chosen from columns 3 to 13 of the Periodic Table may, in one embodiment, improve viscoelastic and mechanical properties of the polymers used in the cosmetic compositions, and for example, may improve hair fixing as well as hair holding time, may improve the cosmetic composition lifting effect, may improve the resistance of the polymers used in nail varnish polish applications, and may improve the ability of the mascara compositions to curve the lashes.

Accordingly, the present disclosure relates to a cosmetic composition comprising, in a cosmetically acceptable medium, at least one compound having an opened or closed cage-like structure, wherein the at least one compound comprises at least 4 atoms of an element chosen from columns 3 to 13 of the Periodic Table, and at least 4 oxygen atoms, wherein the atoms of the element are linked to the oxygen atoms and to at least one substituent, which are the same or different.

As used herein, a “compound having a cage-like structure” means a compound, wherein the atomic arrangement of the element and of the oxygen atoms, and the atomic bonding arrangement of the element and of the oxygen atoms form at least 3 faces, such as 4 faces, of at least one polyhedron; the vertex of the faces are formed with the atoms and the edges of the faces being formed with the bonds.

As used herein, a “closed cage” means a cage wherein the whole polyhedron edges form a bond between an atom of the element and an oxygen atom.

As used herein, an “opened cage” means a cage wherein some polyhedron edges do not form any bond between an atom of the element and an oxygen atom.

For example, the at least one compound comprises from 4 to 20, such as from 4 to 12, further for example 6, 7, 8, 9, 10 or 12 atoms of an element chosen from columns 3 to 13 of the Periodic Table.

In one embodiment, the at least one compound has a closed cage-like structure.

According to at least one embodiment, the cage-like structure of the at least one compound forms a thin sheet.

As used herein, a “thin sheet” means a set of polyhedrons having a 2 by 2 common face and oriented in one plane.

For example, when polyhedrons are cubes, a compound having a cage-like structure forming a thin sheet may have the following geometry:

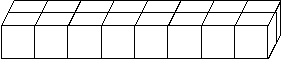

According to at least one embodiment, the cage-like structure of the at least one compound forms a group.

As used herein, a “group” means the stacking of at least two thin sheets in a direction that does not belong to the planes of the thin sheets.

As explained hereinabove, the at least one compound having a cage-like structure comprises at least 4 atoms of an element chosen from columns 3 to 13 of the Periodic Table. The Periodic Table may utilize two group classification systems, which rely on an A/B coding scheme to categorize periodic properties (i.e., VIIA, IIB, etc.). There, however, is the potential for confusion between these two systems. The International Union of Pure and Applied Chemistry (IUPAC) recommends the use of 1-18 group classification based on columns, which is utilized throughout the present disclosure.

For example, the element is chosen from columns 8 to 13, such as from iron, aluminium, and gallium.

When the element is aluminium, the at least one compound is called alumoxane.

Depending on their valency, elements of column 3 to 13 of the Periodic Table may be linked to at least one substituent, which are the same or different.

The at least one substituent may be chosen from hydrogen, groups comprising hydroxyl, alkyl, alkylene, alkenyl, aryl, acyl, alkoxy, such as methoxy, hydride, ester, carboxyl, acrylate, alkyl acrylate, alcohol, aldehyde, amine, alkyl amine and silanol groups, groups comprising at least one amine functionality, siloxane groups, groups comprising at least one siloxane group, silicone groups and groups comprising at least one silicone group, silane groups and groups comprising at least one silane group, groups comprising at least one fluorine, sulfur and phosphor atom, SO₂, CO₂X, SO₃X groups, wherein X is chosen from a hydrogen atom, a methyl and an ethyl group, alpha-olefin groups, epoxide, azo, diazo, halogen, cyclic groups optionally causing a ring opening isomerization, molecular silica, nitrile and thiol groups and polymers, such as polyacrylates, epoxy resins, phenol formaldehyde resins, polyamides, polyesters, polyimides, polycarbonates, polyurethanes, and quinone amine polymers.

Phenol, quaternary amine, haloalkyl, methacrylate, halosilane, styrene and norbornenyl, and tert-butyl groups may be, for example, mentioned.

For example, substituents enabling a better solubilization of the cage-like structured compounds in the physiologically acceptable medium, substituents having an affinity for one hair or skin component, and cationic substituents may be used.

When the at least one substituent comes as a polymer, the polymer may be obtained either by:
polymerizing the cage-like structured compounds, the at least one compound is functionalized by at least two chemically reactive substituents to enable polymerization, or
grafting onto the at least one compound at least one polymeric substituent, such as described herein.

For example, aluminosiloxane based polymers are described in International Patent Application Publication No. WO 00/09578.

When the at least one compound is alumoxane, they may be chosen from the following formulae: [(R)Al(O)]ₙ and R′[(R″)Al(O)]ₙAlR′″₂, wherein R, R′, R″ and R′″ are substituents, such as described herein and n is an integer ranging from 4 to 20.

Similarly, when the at least one compound is gallium based, they may be chosen from the following formulae: [(R)Ga(O)]ₙ and R[(R′)Ga(O)]ₙAlR′″₂, wherein R, R′, R″ and R′″ are substituents, such as described herein and n is an integer ranging from 4 to 20.

For example, the following compounds may be mentioned:
[(^{t}Bu)M(μ₃-O)]₆ having following structure: 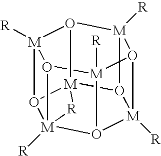
   wherein R is a tert-butyl group,
[(^{t}Bu)M(μ₃-O)]₇ having following structure: 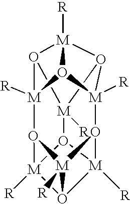
   wherein R is a tert-butyl group,
[(^{t}Bu)M(μ₃-O)]₈ having following structure: 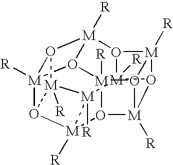
   wherein R is a tert-butyl group,
[(^{t}Bu)M(μ₃-O)]₉ having following structure: 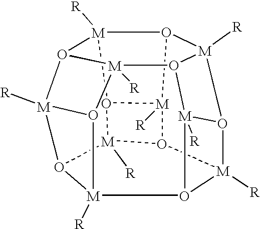
   wherein R is a tert-butyl group,
[(^{t}Bu)M(μ₃-O)]₁ having following structure: 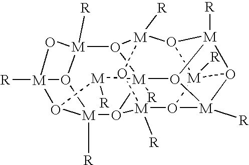
   wherein R is a tert-butyl group,
[(^{t}Bu)M(μ₃-O)]₁₂ having following dodecahedral structure: 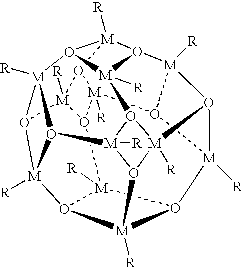
   wherein R is a tert-butyl group,
   M is chosen from Al (i.e., aluminum) and Ga (i.e., gallium).

These compounds are closed cage-like structure compounds.

Examples of opened cage-like structure alumoxane compounds comprise:
opened cage-like structure [^{t}Bu₇Al₅(μ₃-O)₃(μ-OH)₂], of formula: 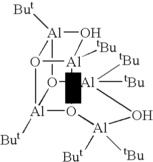
opened cage-like structure [^{t}Bu₈Al₆(μ₃-O)₄(μ-OH)₂], of formula:

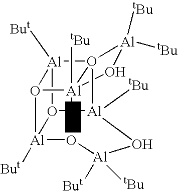

Aluminium base cage synthesis is described, for example, in the following publications: S. Pasynkiewicz, 9 Polyhedron 429-53 (1990), C. T. Vogelson, A. R Barron, 290 Journal of Non-cristalline Solids 216-223 (2001), Callender R. L., C. J. Harlan, N. M. Shapiro, C. D. Jones, D. B. Macqueen, D. L. Callahan, M. R. Wiesner, R. Cook and A. R Barron, 9 Chemistry of Materials 2418-433 (1997).

Iron base cage synthesis is described, for example, in the following publications: J. Rose, M. Cortalezzi-Fidalgo, S. Moustier, C. Magnetto, C. Jones, A. Barron, M. Wiesner, J Y Bottero, 14 Chemistry of Materials 621-28 (2002).

Gallium base cage synthesis is described in: H. W. Roesky, 34 Acc. Chem. Res. 201-11 (2001).

In one embodiment of the cosmetic composition, the at least one compound having a cage-like structure is present in an amount ranging from 0.00001% to 20%, such as from 0.0001% to 10%, further for example, from 0.001% to 5%, by weight relative to the total weight of the composition.

According to the present disclosure, the composition is in a cosmetically acceptable medium.

To this end, at least one solvent is chosen from solvents traditionally used in cosmetic compositions, including water, C₁-C₄ lower alcohols, such as ethanol and isopropanol, C₃-C₆ ketones, such as acetone and methylethylketone, C₃-C₆ esters, such as ethyl acetate and butyl acetate, C₂-C₆ ethers, such as diethoxyethane and dimethoxyethane, and C₆-C₁₀alkanes.

Apart from the at least one cage-like structured compound, the cosmetic composition according to the present disclosure may further comprise at least one cationic, anionic, amphoteric or non ionic fixing polymer.

As used herein, “fixing polymer” means any polymer that may give a shape to the hair or that may retain it.

Cationic fixing polymers to be used according to the present disclosure may be chosen from polymers having primary, secondary, tertiary, and/or quaternary moieties belonging to the polymeric chain or directly bound to it and having a molecular weight ranging from 500 to about 5,000,000 and for example, from 1,000 to 3,000,000.

For example, these polymers may comprise the following cationic polymers:
(1) homopolymers or copolymers derived from acrylic or methacrylic esters or amides and comprising at least one of the following units:

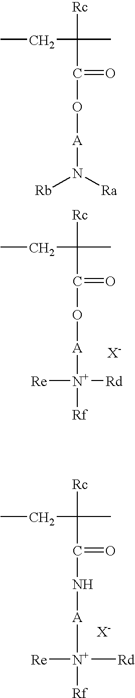

wherein

Ra and Rb, each is chosen from a hydrogen atom and a C₁₋₆ alkyl group, Rc is chosen from a hydrogen atom and a CH₃ radical,

Rd, Re and Rf, which are the same or different, each is chosen from a C₁₋₁₈ alkyl group and a benzyl radical,

A is chosen from a linear and branched C₁₋₆ alkyl group and C₁₋₄ hydroxyalkyl group and

X⁻ is chosen from a methosulfate and a halide anion, such as a chloride or a bromide ion.

Family (1) copolymers may further comprise at least one unit derived from comonomers that may be chosen from acrylamides, methacrylamides, diacetone-acrylamides, acrylamides and methacrylamides substituted on the nitrogen by lower alkyl groups, acrylic and methacrylic acids and esters thereof, vinyl lactames, such as vinyl pyrrolidone and vinyl caprolactame, and vinyl esters.

For example, family (1) copolymers may include:
acrylamide and dimethylaminoethyl methacrylate copolymers quaternized with dimethyl sulfate or with a dimethyl halide, such as those marketed under the trade name HERCOFLOC® by HERCULES,
acrylamide and methacryloyl-oxyethyl trimethylammonium chloride copolymers described, for example, in the European Patent Application No. EP-A-080976 and marketed under the trade name BINA QUAT® P 100 by CIBA GEIGY,
acrylamide and methacryloyl-oxyethyl trimethylammonium methosulfate copolymers marketed under the trade name RETEN® by HERCULES,
vinyl pyrrolidone/dialkylaminoalkyl acrylate or methacrylate copolymers quaternized or not, such as products marketed under the trade name GAFQUAT® by ISP, such as GAF-QUAT® 734 or GAFQUAT® 755, or products called COPOLYMER® 845, 958 and 937. These polymers are described in French Patent Application Nos. FR 2,077,143 and FR 2,393,573,
dimethylaminoethyl methacrylate vinyl caprolactame/vinyl pyrrolidone terpolymers, such as the products marketed under the trade name GAFFIX VC 713 by ISP, and
vinyl pyrrolidone/dimethylaminopropyl methacrylamide quaternized copolymer, such as the products marketed under the trade name GAFQUAT® HS 100 by ISP;

(2) quaternized polysaccharides described, for example, in U.S. Pat. Nos. 3,589,578 and 4,031,307, such as guar gums comprising cationic trialkyl ammonium moieties.

Such products are, for example, marketed under the trade names JAGUAR® C13 S, JAGUAR® C15 and JAGUAR® C17 by MEYHALL;

(3) vinyl pyrrolidone and vinyl imidazole quaternary copolymers, such as the products marketed by BASF under the trade name LUVIQUAT® TFC;

(4) chitosans or salts thereof, such as chitosan acetate, lactate, glutamate, gluconate or pyrrolidone carboxylate.

For example, chitosan may have a 90.5%, by weight deacetylation rate marketed under the trade name KYTAN BRUT STANDARD® by ABER TECHNOLOGIES, chitosan pyrrolidone carboxylate marketed under the trade name KYTAMER® PC by AMERCHOL;

(5) cellulose cationic derivatives, such as cellulose copolymers or cellulose derivatives grafted onto a water-soluble monomer comprising a quaternary ammonium group and described, for example, in U.S. Pat. No. 4,131,576, such as hydroxyalkyl celluloses, hydroxymethylhydroxyethyl or hydroxypropyl celluloses grafted such as onto a methacryloyloxyethyltrimethyl ammonium salt, a methacrylamidopropyltrimethyl ammonium salt or a dimethyldiallyl ammonium salt.

The marketed products corresponding to this definition are, for example, marketed under the trade names CELQUAT® L 200 and CELQUAT® H 100 by NATIONAL STARCH.

Anionic fixing polymers usually employed may be polymers comprising moieties derived from a carboxylic, sulfonic or phosphoric acid and having a weight average molecular weight ranging from about 500 to 5,000,000.

Carboxylic acid moieties may be provided by unsaturated monomers comprising at least one carboxylic acid functionality, such as those having following formula: 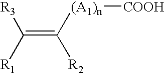
wherein n is an integer ranging from 0 to 10, A is a methylene moiety, optionally linked to the carbon atom of the adjacent unsaturated moiety or methylene moiety, when n is greater than 1, through a heteroatom, such as oxygen or sulfur, R₃ is chosen from a hydrogen atom, a phenyl and benzyl moiety, R₁ is chosen from a hydrogen atom, a lower alkyl and a carboxyl moiety, and R₂ is chosen from a hydrogen atom, a lower alkyl moiety, a —CH₂—COOH, phenyl, and benzyl moiety.

In the above formula, a lower alkyl radical, for example, refers to a moiety having from 1 to 4 carbon atoms such as a methyl or an ethyl group.

For example, the carboxyl group comprising anionic fixing polymers according to the present disclosure are as follows:

A) acrylic or methacrylic acid homo- or copolymers or salts thereof such as the products marketed under the trade names VERSICOL® E or K by ALLIED COLLOID, and under the trade name ULTRAHOLD® by BASF; acrylic acid and acrylamide copolymers sold as sodium salt under the trade names RETEN® 421, 423 or 425 by HERCULES; sodium salts of polyhydroxycarboxylic acids.

B) copolymers of acrylic acid or methacrylic acid with a monoethylenic monomer, such as ethylene, styrene, vinyl esters, acrylic or methacrylic acid esters.

These copolymers may be grafted onto a polyalkylene glycol, such as polyethylene glycol and may optionally be crosslinked.

Such polymers are, for example, described in French Patent Application No. FR 1,222,944 and in German Patent Application No. DE 2,330,956. Copolymers may be mentioned that comprise an optionally N-alkylated and/or hydroxyalkylated acrylamide unit in their chain, such as those described in the Luxemburger Patent Application Nos. LU 75370 and LU75371 or proposed under the trade name QUADRAMER® by AMERICAN CYANAMID.

Acrylic acid and C₁-C₄ alkyl methacrylate copolymers, as well as vinyl pyrrolidone, (meth)acrylic acid and C₁-C₂₀ alkyl (meth)acrylate terpolymers, for example, based on lauryl (ACRYLDONE® LM from ISP), tert-butyl (LUVIFLEX® VBM 70 marketed by BASF) or methyl (STEPANHOLD® EXTRA marketed by STEPAN), and methacrylic acid/ethyl acrylate/tert-butyl acrylate terpolymers, such as the products marketed under the trade name LUVIMER® 100 P by BASF may also be mentioned.

C) Copolymers derived from crotonic acid, such as those comprising vinyl acetate or vinyl propionate units in their chain, and optionally other monomers, such as allyl, methallyl or vinyl esters of a saturated, linear or branched, carboxylic acid, with a hydrocarbyl long chain comprising at least 5 carbon atoms, these polymers being optionally grafted and cross-linked, or vinyl, allyl or methallyl esters of a α- or β-cyclic carboxylic acid.

Such polymers are described, inter alia, in the French Patent Application Nos. FR 1,222,944, FR 1,580,545, FR 2,265,782, FR 2,265,781, FR 1,564,110, and FR 2,439,798.

Resins 28-29-30, 26-13-14 and 28-13-10 marketed by NATIONAL STARCH are further examples of commercial products belonging to this class.

D) Copolymers derived from C₄-C₈ mono-unsaturated carboxylic acids or anhydrides may be chosen from:
copolymers comprising:
(ii) at least one maleic, fumaric, itaconic acid(s) or anhydride(s), and
(iii) at least one monomer chosen from vinyl esters, vinyl ethers, vinyl halides, phenyl vinyl derivatives, acrylic acid and esters thereof, and anhydride functionalities of these copolymers being optionally monoesterified or monoamidified.

Such polymers are, for example, described in U.S. Pat. Nos. 2,047,398, 2,723,248, 2,102,113, and United Kingdom Patent No. GB 839,805 and for example, those marketed under the trade names GANTREZ AN or ES, AVANTAGE® CP by ISP;
copolymers comprising (i) at least one maleic, citraconic or itaconic anhydride(s) and (ii) at least one monomer chosen from allyl or methallyl esters comprising optionally at least one acrylamide, methacrylamide, α-olefin, acrylic or methacrylic ester, acrylic or methacrylic acid or vinyl pyrrolidone moiety(ies) in their chain, and the anhydride functionalities of these copolymers being optionally monoesterified or monoamidified.

These polymers are, for example, described in the French Patent Application Nos. FR 2,350,384 and FR 2,357,241 of the applicant.

E) Polyacrylamides comprising carboxylate moieties.

The anionic moieties of the anionic fixing polymers of the present disclosure may also be sulfonic acid groups provided by vinylsulfonic, styrenesulfonic, naphtalenesulfonic or acrylamidoalkylsulfonic units.

These sulfonic acid group comprising polymers may be chosen:
poly(vinylsulfonic acid) salts having a weight average molecular weight ranging from about 1000 to 100,000, as well as copolymers of vinylsulfonic acid with an unsaturated comonomer, such as acrylic acid, methacrylic acid, esters of these acids, acrylamide, acrylamide derivatives, vinyl ethers, and vinyl pyrrolidone;
poly(styrenesulfonic acid) salts. Mention may be made, for example, of two sodium salts having a weight average molecular weight of about 500,000 to about 100,000, respectively marketed under the trade names FLEXAN® 500 and FLEXAN® 130 by NATIONAL STARCH. These compounds are described in the French Patent No. FR 2,198,719;
poly(acrylamidesulfonic acid) salts, such as those mentioned in U.S. Pat. No. 4,128,631 and further for example, the poly(acrylamidoethylpropanesulfonic acid) marketed under the trade name COSMEDIA POLYMER® HSP 1180 by HENKEL.

According to the present disclosure, anionic fixing polymers may be, for example, chosen from: acrylic acid copolymers, such as acrylic acid/ethyl acrylate/N-tert-butylacrylamide terpolymers marketed, for example, under the trade name ULTRAHOLD STRONG® by BASF; crotonic acid derived copolymers, such as vinyl acetate/vinyl tert-butyl benzoate/crotonic acid terpolymers, and crotonic acid/vinyl acetate/vinyl neododecanoate terpolymers marketed, for example, under the trade name Résine 28-29-30 by NATIONAL STARCH; maleic, fumaric and itaconic acid and anhydride derived copolymers comprising as comonomers vinyl esters, vinyl ethers, vinyl halides, phenyl vinyl derivatives, acrylic acid and acrylic acid esters, such as methylvinyl ether/monoesterified maleic anhydride copolymers, marketed, for example, under the trade name GANTREZ® by ISP; methacrylic acid and methyl methacrylate copolymers marketed under the trade name EUDRAGIT®L by ROHM PHARMA; methacrylic acid/methyl methacrylate/C₁₋₄ alkyl acrylate/acrylic acid and C₁₋₄ hydroxyalkyl methacrylate copolymers marketed under the trade name AMERHOLD® DR 25 by AMERCHOL, or under the trade name ACUDYNE® 255 by ROHM & HAAS; methacrylic acid and ethyl acrylate copolymers marketed under the trade name LUVIMER® MAEX or MAE by BASF; and vinyl acetate/crotonic acid copolymers and vinyl acetate/crotonic acid copolymers grafted with polyethylene glycol marketed under the trade name ARISTOFLEX® A by BASF.

Amphoteric fixing polymers to be used in the present disclosure may be, for example, chosen from the polymers comprising B and C units statistically distributed in the polymeric chain, wherein B is a unit derived from a monomer comprising at least one basic nitrogen atom and C is a unit derived from a monomer comprising at least one carboxylic acid or sulfonic acid moiety(ies). Amphoteric fixing polymers may also comprise zwitterionic units of carboxybetaine or sulfobetaine type. There also may be polymers with a cationic main chain comprising primary, secondary, tertiary or quaternary amine moieties, amongst which at least one bears a carboxylic acid or sulfonic acid moiety through a hydrocarbyl radical. Amphoteric fixing polymers may further have an anionic chain derived from α,β-unsaturated dicarboxylic acids, one carboxyl moiety of which has been reacted with a polyamine comprising at least one primary or secondary amine moiety.

Amphoteric fixing polymers corresponding to the herein definition may be, for example, chosen from the following polymers:
(1) Polymers resulting from the copolymerization of a vinyl monomer bearing a carboxylic acid moiety, such as acrylic acid, methacrylic acid, maleic acid, α-chloroacrylic acid, and of a vinyl monomer comprising at least one basic functionality, such as dialkylaminoalkyl methacrylate and -acrylate or dialkylaminoalkyl (meth)acrylamides. Such compounds are described, for example, in U.S. Pat. No. 3,836,537.
(2) Polymers comprising units derived from:
   (a) at least one monomer chosen from t N-alkylated acrylamides and methacrylamides,
   (b) at least one comonomer comprising at least one carboxylic acid functionality, and
   (c) at least one basic comonomer, such as esters substituted with a primary, secondary, tertiary or quaternary amine of acrylic acid and methacrylic acid, and the quaternization product of dimethylaminoethyl methacrylate and dimethyl- or diethylsulfate.

For example, mention may be made to monomers (a) of: N-alkylated acrylamides or methacrylamides bearing C₂₋₁₂ alkyl radicals, such as N-ethylacrylamide, N-tert-butylacrylamide, N-tert-octylacrylamide, N-octylacrylamide, N-decylacrylamide, N-dodecylacrylamide as well as corresponding methacrylamides.

Carboxylic acid group comprising comonomers (b) may, for example, be chosen from acrylic, methacrylic, crotonic, itaconic, maleic, fumaric acids as well as from C₁₋₄ alkyl monoesters of maleic and fumaric acids and anhydrides.

Mention may be made to comonomers (c) of aminoethyl methacrylate, butylaminoethyl methacrylate, N,N′-dimethylaminoethyl methacrylate and N-tert-butylaminoethyl methacrylate.

For example, employed may be copolymers which CTFA name (4^{th} Ed., 1991) is “Octylacrylamide/acrylates/butylaminoethyl methacrylate copolymer”, such as the products marketed under the trade name AMPHOMER® or LOVOCRYL® 47 by NATIONAL STARCH.

(3) Cross-linked and alkylated polyaminoamides, fully or partly derived from polyaminoamides of general formula:
—[C(═O)—R₄—C(═O)-Z-]- (II)

wherein R₄ is chosen from a divalent radical derived from a saturated dicarboxylic acid, a mono- and dicarboxylic aliphatic acid with ethylene double bond, a C₁₋₆ alkyl ester of these acids, and a radical resulting from the addition of anyone of the acids onto a bis-primary or bis-secondary amine, and Z is chosen from a radical of a bis-primary, mono- and bis-secondary polyalkylene-polyamine, and for example, is chosen from:

a) in an amount ranging from 60 mol % to 100 mol %, the radical:
—NH—[(CH₂)ₓ—NH]ₚ— (3)

wherein x=2 and p=2 or 3, or x=3 and p=2,

this radical being derived from diethylene triamine, triethylene tetraamine or dipropylene triamine;

b) in an amount ranging from 0 mol % to 40 mol %, the radical of formula (3), wherein x=2 and p=1, and wherein derived from ethylene diamine, or the radical derived from piperazine:

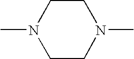

c) in an amount ranging from 0 mol % to 20 mol %, the radical —NH—(CH₂)₆—NH— derived from hexamethylene diamine, these polyaminoamines being cross-linked by adding from 0.025 mol to 0.35 mol of the amine moiety of a difunctional cross-linking agent chosen from epihalohydrines, diepoxides, dianhydrides, and di-unsaturated compounds alkylated with acrylic acid, chloroacetic acid or with an alkane-sulfone.

The saturated carboxylic acids are, for example, chosen from acids having from 6 to 10 carbon atoms, such as adipic acid, trimethyl-2,2-4-adipic acid and trimethyl-2,4,4-adipic acid, terephtalic acid, ethylene double bond acids, for example, acrylic, methacrylic and itaconic acids.

The alkane-sulfones used for alkylation are, for example, propane sulfone or butane sulfone.

Alkylation agent salts are, for example, sodium or potassium salts.

(4) Polymers comprising zwitterionic units of formula:

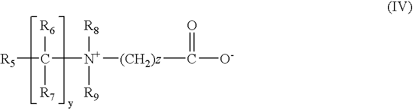

wherein R₅ is a polymerizable unsaturated moiety, such as an acrylate, methacrylate, acrylamide or methacrylamide moiety, y and z each are each an integer ranging from 1 to 3, R₆ and R₇ each independently is chosen from a hydrogen atom and a methyl, ethyl and propyl group, R₈ and R₉ each independently is chosen from a hydrogen atom, and an alkyl radical, wherein the total number of carbon atoms in R₈ and R₉ does not exceed 10.

The polymers comprising such units of formula (IV) may further comprise units derived from non zwitterionic monomers, such as dimethyl or diethylamino ethyl acrylate or methacrylate, alkyl acrylates or methacrylates, acrylamides or methacrylamides or vinyl acetate.

By way of example, mention may be made of the copolymer of methyl methacrylate/dimethyl carboxymethyl ammonioethyl methacrylate copolymer, such as the product marketed under the trade name DIAFORMER® Z301 by SANDOZ.

(5) Chitosan derived polymers comprising monomer units of following formulae:

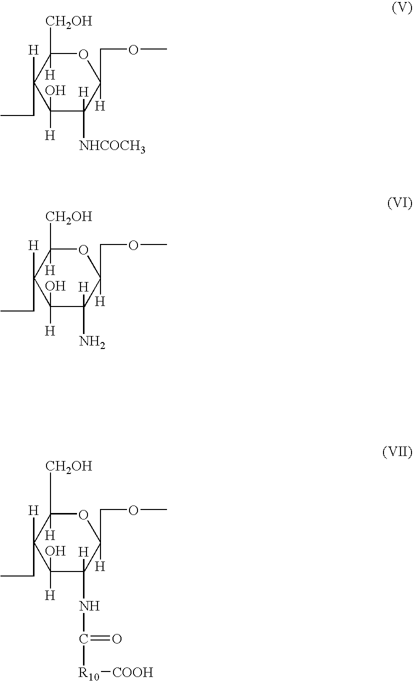

The formula (V) unit may be present in an amount ranging from 0% to 30%, the formula (VI) unit may be present in an amount ranging from 5% to 50%, and the formula (VII) unit, may be present in an amount ranging from 30% to 90%, wherein in formula (VII), R₁₀ is chosen from a radical of formula:

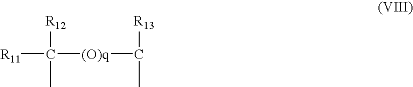

wherein:

if q=0, then R₁₁, R₁₂ and R₁₃, which may be the same or different, each is chosen from a hydrogen atom, a methyl, hydroxyl, acetoxy and amino group, a monoalkyl amine and dialkyl amine group optionally interrupted by at least one nitrogen atom and/or optionally substituted by at least one amine, hydroxyl, carboxyl, alkylthio or sulfo group, wherein the group alkylthio, the alkyl group bears an amino radical, at least one of the radicals R₁₁, R₁₂ and R₁₃ being in that case a hydrogen atom; or

if q=1, then R₁₁, R₁₂ and R₁₃ each is chosen from a hydrogen atom, as well as salts formed by these compounds with bases or acids.

6) Polymers obtained by chitosan N-carboxyalkylation, such as N-carboxymethylchitosan or N-carboxybutylchitosan marketed under the trade name EVALSAN® by JAN DEKKER.

(7) Polymers having following general formula (IX):

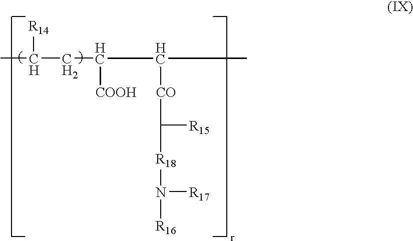

For example, those described in the French Patent Application No. FR 1,400,366, wherein R₁₄ is chosen from a hydrogen atom and a CH₃O, CH₃CH₂O and phenyl radical, R₁₅ is chosen from a hydrogen atom and a lower alkyl radical, such as methyl or ethyl, R₁₆ is chosen from a hydrogen atom and a lower alkyl radical, such as methyl or ethyl, R₁₇ is chosen from a lower alkyl radical, such as methyl or ethyl or a radical having following formula:
—R₁₈—N(R₁₆)₂,

wherein R₁₈ is chosen from a —CH₂—CH₂—, —CH₂—CH₂—CH₂—, —CH₂—CH(CH₃)— moiety and R₁₆ is chosen from, such as defined herein, as well as higher homologs of these radicals comprising up to 6 carbon atoms.

(8) Amphoteric polymers of -D-X-D-X- type chosen from:

(a) polymers obtained by reacting chloroacetic acid or sodium chloroacetate with the compounds comprising at least one unit of formula:
-D-X-D-X-D- (X)
wherein D is a radical: 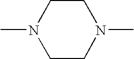
and X is chosen from the symbol E or E′, E or E′, which may be the same or different, each is chosen from a divalent radical that is a straight or branched chain alkylene radical comprising up to 7 carbon atoms in the main chain, which is unsubstituted or substituted with hydroxyl moieties and that may further comprise oxygen, nitrogen or sulfur atoms, from 1 to 3 aromatic and/or heterocyclic rings; the oxygen, nitrogen and sulfur atoms being present in the form chosen from ether, thioether, sulfoxide, sulfone, sulfonium, alkyl amine, alkenyl amine moieties, hydroxyl, benzyl amine, amine oxide, quaternary ammonium, amide, imide, alcohol, ester and urethane moieties.

b) Polymers of following formula:
-D-X′-D-X′- (X′)
wherein D is a radical

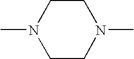

and X′ is chosen from the symbol E or E′ and at least one time E′, E having the meaning given above and E′ is chosen from a divalent radical that is a straight or branched chain alkylene radical having up to 7 carbon atoms in the main chain, which is substituted or unsubstituted with at least one hydroxyl radical and comprising at least one nitrogen atom, the nitrogen atom being substituted by an alkyl chain optionally interrupted with an oxygen atom and necessarily comprising at least one carboxyl functionality or at least one hydroxyl functionality, and being betainized by reacting with chloroacetic acid or sodium chloroacetate.

9) (C₁₋₅)alkyl vinyl ether/maleic anhydride copolymers partially modified by semi-amidification with a N,N-dialkylaminoalkylamine, such as N,N-dimethylaminopropylamine, or by semi-esterification with a N,N-dialkanolamine. These copolymers may also comprise other vinyl comonomers, such as vinyl caprolactame.

Of the amphoteric fixing polymers according to the present disclosure described above, mention may be made, for example, to those of family (3), such as those whose CTFA name is “Octylacrylamide/acrylates/butylaminoethyl-methacrylate copolymer”. Mention may also be made, for example, of the products marketed under the trade names AMPHOMER®, AMPHOMER® LV 71 or LOVOCRYL® 47 by NATIONAL STARCH.

Other amphoteric fixing polymers that may be mentioned are those of family (4), of methyl methacrylate and dimethylcarboxymethyl ammonioethyl methacrylate copolymers, marketed, for example, under the trade name DIAFORMER® Z301 by SANDOZ.

The anionic or amphoteric fixing polymers may, if necessary, be partially or fully neutralized. Neutralizing agents may be, for example, soda, potash, amino-2-methylpropanol, monoethanolamine, triethanolamine or tri-isopropanolamine, inorganic or organic acids, such as hydrochloric acid or citric acid.

The non ionic fixing polymers that may be used according to the present disclosure are, for example, chosen from:
vinyl pyrrolidone homopolymers,
vinyl pyrrolidone and vinyl acetate copolymers,
polyalkyl oxazolines, such as polyethyl oxazolines by DOW CHEMICAL under the trade names PEOX® 50 000, PEOX® 200 000 and PEOX® 500 000,
vinyl acetate homopolymers, such as those products under the trade name APPRETAN® EM by HOECHST or the product proposed under the trade name RHODOPAS® A 012 by RHONE POULENC;
vinyl acetate and acrylic ester copolymers, such as those products under the trade name RHODOPAS® AD 310 by RHONE POULENC,
vinyl acetate and ethylene copolymers, such as those products under the trade name APPRETAN® TV by HOECHST,
vinyl acetate and maleic ester copolymers, for example, based on dibutyl maleate, such as those products under the trade name APPRETAN® MB EXTRA by HOECHST,
ethylene and maleic anhydride copolymers,
alkyl acrylate homopolymers and alkyl methacrylate homopolymers, such as those products under the trade name MICROPEARL® RQ 750 by MATSUMOTO or the product under the trade name LUHYDRAN® A 848 S by BASF,
acrylic ester copolymers, such as alkyl acrylate and alkyl methacrylate copolymers, such as those products by ROHM & HAAS under the trade names PRIMAL AC-261 K and EUDRAGIT NE 30 D, by BASF under the trade names ACRONAL® 601, LUHYDRAN® LR 8833 or 8845, and by HOECHST under the trade names APPRETAN® N 9213 or N 9212,
copolymers of acrylonitrile with a non ionic monomer chosen, for example, from butadiene and alkyl (meth)acrylates, such as the products under the trade names NIPOL® LX 531 B by NIPPON ZEON or those under the trade name CJ 0610 B by ROHM & HAAS,
polyurethanes, such as the products under the trade names ACRYSOL® RM 10200R ACRYSOL® RM 2020 by ROHM & HMS, the products URAFLEX® XP 401 UZ, URAFLEX® XP and 402 UZ by DSM RESINS,
alkyl acrylate and urethane copolymers, such as the products 8538-33 marketed by NATIONAL STARCH,
polyamides, such as the products ESTAPOR® LO 11 by RHONE POULENC,
non ionic guar gums, chemically modified or not modified.

Non ionic, non modified guar gums are, for example, the products marketed under the trade name VIDOGUM® GH 175 by UNIPECTINE and under the trade name JAGUAR® C by MEYHALL. Non ionic, modified guar gums that may be used according to the present disclosure are, for example, modified by C₁₋₆ hydroxyalkyl moieties. Hydroxymethyl, hydroxyethyl, hydroxypropyl and hydroxybutyl moieties may be examples thereof.

These guar gums are well known in the art and may, for example, be prepared by reacting corresponding alkene oxides, such as propylene oxides, with the guar gum to obtain a hydroxypropyl moieties modified guar gum.

Such non ionic guar gums, optionally modified with hydroxyalkyl moieties are, for example, marketed under the trade names JAGUARS HP8, JAGUAR® HP60 and JAGUARS HP120, JAGUARS DC 293 and JAGUARS HP 105 by MEYHALL, or under the trade name GALACTASOL® 4H4FD2 by AQUALON.

According to the present disclosure, graft silicone-type film-forming polymers may also be used as fixing polymers, comprising a polysiloxane part and a part made of a non silicone organic chain, one of the two parts forming the polymer main chain and the other one being graft onto said main chain.

These polymers are described, for example, in European Patent Application Nos. EP-A-0,412,704, EP-A-0,412,707, EP-A-0,640,105 and International Patent Application Publication No. WO 95/00578, European Patent Application No. EP-A-0,582,152 and International Patent Application Publication No. WO 93/23009, and in U.S. Pat. Nos. 4,693,935, 4,728,571, and 4,972,037.

These polymers may be, for example, anionic or non ionic in nature.

Such polymers are, for example, copolymers that may be prepared by free radical polymerization from the monomer mixture comprising,

a) from 50% to 90%, by weight of tert-butyl acrylate,

b) from 0% to 40%, by weight of acrylic acid,

c) from 5% to 40%, by weight of a silicone macromer of following formula: 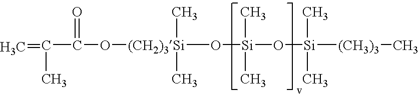
wherein v is a number ranging from 5 to 700, and wherein the weight percentages are based on the monomer total weight.

Other examples of silicone graft polymers may be polydimethyl siloxanes (PDMS), onto which mixed polymer units of poly(meth)acrylic type and alkyl poly(meth)acrylate type are grafted through a chain linkage of the thiopropylene type, and polydimethyl siloxanes (PDMS) onto which polymer units of the isobutyl poly(meth)acrylate type are grafted through a chain linkage of the thiopropylene type.

Functionalized or non-functionalized, silicone or non silicone, polyurethanes may also be used as film-forming fixing polymers.

Polyurethanes that may be, for example, used with the present disclosure are those described in Patent Application Nos. EP 0,751,162, EP 0,637,600, FR 2,743,297 and EP 0,648,485 of the applicant, as well as in Patent Application Nos. EP 0,656,021 or WO 94/03510 from BASF and EP 0,619,111 from NATIONAL STARCH.

The cosmetic composition according to the present disclosure may further comprise at least one cosmetically acceptable additives chosen from silicones in a soluble, dispersed, micro- and nanodispersed form, thickening agents, non ionic, anionic, cationic and amphoteric surfactants, conditioning agents, softening agents, anti-foaming agents, moisturizing agents, emollients, plasticizers, water-soluble and oil-soluble, silicone and non silicone sunscreens, permanent and temporary dyes, organic and inorganic pigments, colored and non-colored, inorganic fillers, clays, nacres and perlescent agents, opacifying agents, colloids, fragrances, peptizing agents, preserving agents, ceramides and pseudo-ceramides, vitamins and provitamins amongst which panthenol, proteins, sequestering agents, solubilizing agents, acidifying agents, alkalizing agents, anti-corrosion agents, fats, such as vegetal, animal, mineral and synthetic oils, reducing agents and antioxidants, oxidants and polymers other than fixing polymers.

Glycerine may be, for example, mentioned as humectant. Synthetic oils also include cyclomethicones, such as pentacyclomethicone.

The cosmetic composition according to the present disclosure may be used as a composition rinsed off or as left-on.

When the cosmetic composition is to be applied onto the hair, it may come as a lotion, a spray, a foam, a wax, a gel, a paste, a shampoo or an after-shampoo (conditioner) or a hair repair product, a perm composition, a hair smoothing product, permanent or not, or as a direct colouring or oxidizing product, a defrizzing product or as a hair bleaching composition.

When the cosmetic composition is to be applied onto the skin, it may come as a skin care composition, such as a cleansing or a make-up remover composition, for example, a lotion, a gel, a foam, a sun cream, a face cream, a mask, a cleansing gel, an oil, a lipstick, an eye-liner, a mascara, a powder, or a eye shadow.

The cosmetic composition according to the present disclosure may be in a form chosen from oil-in-water emulsion and a water-in-oil emulsion.

When the cosmetic composition according to the present disclosure comes as an emulsion, it comprises typically at least one non ionic, anionic, cationic or amphoteric surfactant.

Emulsifying agents may be suitably chosen depending on the targeted emulsion, e.g., either a water-in-oil (W/O) emulsion or a oil-in-water emulsion (O/W).

For the oil-in-water emulsions (O/W), examples of suitable emulsifying agents may include:
as amphoteric emulsifying agents, N-acyl-aminoacids, such as N-alkylaminoacetates and disodium cocoamphodiacetate, and amine oxides, such as stearamine oxide;
as anionic emulsifying agents, acyl-glutamates, such as “disodium hydrogenated tallow glutamate” (Amisoft HS-21® marketed by AJINOMOTO); carboxylic acids and salts thereof, such as sodium stearate; phosphoric esters and salts thereof, such as “DEA oleth-10 phosphate”; sulfosuccinates, such as “disodium PEG-5 citrate lauryl sulfosuccinate” and “disodium ricinoleamido MEA sulfosuccinate”;
as cationic emulsifying agents, alkyl-imidazolidinium, such as isostearyl-ethylimidonium ethosulfate; ammonium salts, such as N,N,N-trimethyl-1-docosanaminium chloride (behentrimonium chloride);
as non ionic emulsifying agents, esters and ose ethers, such as sucrose stearate, sucrose cocoate and the sorbitan stearate and sucrose cocoate mixture marketed by ICI under the trade name Arlatone 2121®; polyol esters, for example glycerol or sorbitol esters, such as glyceryl stearate, polyglyceryl-2 stearate, sorbitan stearate; glycerol ethers; oxyethylenated and/or oxypropylenated ethers, such as oxyethylenated, oxypropylenated ether of lauric alcohol with 25 oxyethylene groups and 25 oxypropylene groups (CTFA name “PPG-25 laureth-25”) and oxyethylenated ether of the C₁₂-C₁₅ fatty alcohol mixture with 7 oxyethylene groups (CTFA name “C₁₂-C₁₅ Pareth-7”); ethylene glycol polymers, such as PEG-100, and mixtures thereof.

At least one such emulsifying agent may be employed.

For water-in-oil emulsions (W/O), suitable emulsifying agent, for example, may include, polyol fatty esters, such as glycerol or sorbitol fatty esters and for example, polyol isostearic, oleic and ricinoleic esters, such as the petrolatum, polyglyceryl-3 oleate, glyceryl isostearate, hydrogenated castor oil and ozokerite mixture, marketed under the trade name Protegin W® by GOLDSCHMIDT, sorbitan isostearate, polyglyceryl di-isostearate, polyglyceryl-2 sesqui-isostearate; ose esters and ethers, such as “methyl glucose dioleate”; fatty esters, such as magnesium lanolate; dimethicone copolyols and alkyl-dimethicone copolyols, such as laurylmethicone copolyol marketed under the trade name Dow Corning 5200 Formulation Aid by DOW CORNING, cetyl dimethicone copolyol marketed under the trade name Abil EM 90® by GOLDSCHMIDT and the dimethicone copolyol marketed under the trade name KF-6015 by SHINETSU, and mixtures thereof.

The cosmetic composition according to the present disclosure may be packaged in aerosol container. If so, it comprises at least one propellant.

The composition according to the present disclosure may be used to shape the hair.

For example, a method for shaping hair comprising applying to the hair a cosmetic composition comprising, in a cosmetically acceptable medium, at least one compound having an opened or a closed cage-like structure, wherein the at least one compound comprises at least 4 atoms of an element chosen from columns 3 to 13 of the Periodic Table, and at least 4 oxygen atoms, wherein the atoms of the element are linked to the oxygen atoms and to at least one substituent, which are the same or different.

One aspect of the present disclosure is to provide the use of the cosmetic composition of at least one cage-like structure compound, such as previously defined, to improve the lifting effect of the composition comprising it.

For example, a method for improving the lifting effect of a cosmetic composition comprising incorporating in the cosmetic composition at least one compound having an opened or a closed cage-like structure, wherein the at least one compound comprises at least 4 atoms of an element chosen from columns 3 to 13 of the Periodic Table, and at least 4 oxygen atoms, wherein the atoms of the element are linked to the oxygen atoms and to at least one substituent, which are the same or different.

Another aspect of the present disclosure is to provide the use of the cosmetic composition comprising at least one polymer of at least one cage-like structure compound, such as previously defined, to improve the mechanical resistance of said polymer(s).

For example, a method for improving the mechanical resistance of a polymer comprising incorporating in the polymer at least one compound having an opened or a closed cage-like structure, wherein the at least one compound comprises at least 4 atoms of an element chosen from columns 3 to 13 of the Periodic Table, and at least 4 oxygen atoms, wherein the atoms of the element are linked to the oxygen atoms and to at least one substituent, which are the same or different.

At least one aspect of the present disclosure is to provide the use in a cosmetic mascara composition of at least one cage-like structure compound, such as previously defined, to improve the lash curving effect.

For example, a method for improving lash curling effect comprising applying to the lashes a cosmetic composition comprising, in a cosmetically acceptable medium, at least one compound having an opened or a closed cage-like structure, wherein the at least one compound comprises at least 4 atoms of an element chosen from columns 3 to 13 of the Periodic Table, and at least 4 oxygen atoms, wherein the atoms of the element are linked to the oxygen atoms and to at least one substituent, which are the same or different.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The examples below serve to illustrate the present disclosure in a non-limiting manner.

### EXAMPLE 1

### Hair Styling Lotion Spray

| | |
|---|---|
| Alumoxane cage-like structure compound, substituted with | 1.0% |
| carboxylic moieties and having a closed cubic structure | |
| Butyl acrylate/acrylic acid/methacrylic acid copolymer | 3.0% |
| Ethanol | 20.0% |
| Water | 76.0% |

The cage-like structure compound used is described in the following publication: Callender R. L., C. J. Harlan, N. M. Shapiro, C. D. Jones, D. B. Macqueen, D. L. Callahan, M. R. Wiesner, R. Cook and A. R Barron, 9 Chemistry of Materials 2418-433 (1997).

### EXAMPLE 2

### Hair Styling Lotion Spray

| | |
|---|---|
| Iron based cage-like structure compound, substituted | 1.0% |
| with carboxylic moieties | |
| Butyl acrylate/acrylic acid/methacrylic acid copolymer | 3.0% |
| Ethanol | 20.0% |
| Water | 76.0% |

The cage-like structure compound used is described in the following publication: J. Rose, M. Cortalezzi-Fidalgo, S. Moustier, C. Magnetto, C. Jones, A. Barron, M. Wiesner, J Y Bottero, 14 Chemistry of Materials 621-28 (2002).

### EXAMPLE 3

### Skin Care Fluid

1. Water phase

| | |
|---|---|
| Aluminium based cage-like structure compound, substituted with | 1.0% |
| carboxylic moieties | |
| Sodium chloride | 2.50% |
| Glycerin | 7.00% |
| Water | 70.00% |

The cage-like structure compound used is described in the following publication: Callender R. L., C. J. Harlan, N. M. Shapiro, C. D. Jones, D. B. Macqueen, D. L. Callahan, M. R. Wiesner, R. Cook and A. R Barron, 9 Chemistry of Materials 2418-433 (1997).

2. Oil phase

| | |
|---|---|
| Dimethicone copolyol | 1.75% |
| (marketed under trade reference KF-6015 by SHINETSU) | |
| Pentacyclomethicone | 17.75% |

What is claimed is:

1. A cosmetic composition comprising, in a cosmetically acceptable medium, at least one compound having an opened or a closed cage-like structure, wherein the at least one compound comprises at least 4 atoms of an element chosen from columns 3 to 13 of the Periodic Table, and at least 4 oxygen atoms, wherein the atoms of the element are linked to the oxygen atoms and to at least one substituent, which are the same or different.

2. The composition according to claim 1, wherein the at least one compound comprises from 4 to 20, atoms of the element.

3. The composition according to claim 2, wherein the at least one compound comprises from 4 to 12, atoms of the element.

4. The composition according to claim 2, wherein the at least one compound comprises 6, 7, 8, 9, 10 or 12, atoms of the element.

5. The composition according to claim 1, wherein the at least one compound has a closed cage-like structure.

6. The composition according to claim 1, wherein the cage-like structure of the at least one compound forms a thin sheet.

7. The composition according to claim 1, wherein the cage-like structure of the at least one compound forms a group.

8. The composition according to claim 1, wherein the element is chosen from columns 8 to 13 of the Periodic Table.

9. The composition according to claim 8, wherein the element is chosen from iron, aluminium, and gallium.

10. The composition according to claim 1, wherein the at least one substituent is chosen from hydrogen, hydroxyl, alkyl, alkylene, alkenyl, aryl, acyl and alkoxy groups, silanol groups, groups comprising at least one amine functionality, siloxane groups, groups comprising at least one siloxane group, silicone groups or groups comprising at least one silicone group, silane groups and groups comprising at least one silane group, groups comprising at least one fluorine, sulfur and phosphor atom, SO₂, CO₂X, SO₃X groups, wherein X is chosen from a hydrogen atom, a methyl and an ethyl group, alpha-olefin groups, epoxide, azo, diazo, halogen, cyclic groups optionally causing a ring opening isomerization, molecular silica, nitrile and thiol groups and polymers, epoxy resins, phenol formaldehyde resins, polyamides, polyesters, polyimides, polycarbonates, polyurethanes, and quinone amine polymers.

11. The composition according to claim 10, wherein the at least one substituent is chosen from hydroxyl, alkyl, phenol, quaternary amine, haloalkyl, methacrylate, halosilane, styrene, norbornenyl, and tert-butyl groups.

12. The composition according to claim 1, wherein the at least one compound is chosen from [(^{t}Bu)M(μ₃-O)]₆, [(^{t}Bu)M(μ₃-O)]₇, [(^{t}Bu)Al(μ₃-O)]₈, [(^{t}Bu)M(μ₃-O)]₉, [(^{t}Bu)M(μ₃-O)]₁₀, and [(^{t}Bu)M(μ₃-O)]₁₂, wherein M is chosen from aluminium and gallium.

13. The composition according to claim 1, wherein the at least one compound is present in an amount ranging from 0.00001% to 20%, by weight relative to the total composition.

14. The composition according to claim 13, wherein the at least one compound is present in an amount ranging from 0.0001% to 10%, by weight relative to the total composition.

15. The composition according to claim 14, wherein the at least one compound is present in an amount ranging from 0.001% to 5%, by weight relative to the total composition.

16. The composition according to claim 1, further comprising a solvent chosen from water, C₁-C₄ lower alcohols, C₃-C₆ ketones, C₃-C₆ esters, C₂-C₆ ethers, and C₆-C₁₀ alkanes.

17. The composition according to claim 1, further comprising at least one fixing polymer.

18. The composition according to claim 1, wherein the composition is in a form chosen from an oil-in-water emulsion and a water-in-oil emulsion.

19. A method for shaping hair comprising applying to the hair a cosmetic composition comprising, in a cosmetically acceptable medium, at least one compound having an opened or a closed cage-like structure, wherein the at least one compound comprises at least 4 atoms of an element chosen from columns 3 to 13 of the Periodic Table, and at least 4 oxygen atoms, wherein the atoms of the element are linked to the oxygen atoms and to at least one substituent, which are the same or different.

20. A method for improving the lifting effect of a cosmetic composition comprising incorporating in the cosmetic composition at least one compound having an opened or a closed cage-like structure, wherein the at least one compound comprises at least 4 atoms of an element chosen from columns 3 to 13 of the Periodic Table, and at least 4 oxygen atoms, wherein the atoms of the element are linked to the oxygen atoms and to at least one substituent, which are the same or different.

21. A method for improving the mechanical resistance of a polymer comprising incorporating in the polymer at least one compound having an opened or a closed cage-like structure, wherein the at least one compound comprises at least 4 atoms of an element chosen from columns 3 to 13 of the Periodic Table, and at least 4 oxygen atoms, wherein the atoms of the element are linked to the oxygen atoms and to at least one substituent, which are the same or different.

22. A method for improving lash curling effect comprising applying to the lashes a cosmetic composition comprising, in a cosmetically acceptable medium, at least one compound having an opened or a closed cage-like structure, wherein the at least one compound comprises at least 4 atoms of an element chosen from columns 3 to 13 of the Periodic Table, and at least 4 oxygen atoms, wherein the atoms of the element are linked to the oxygen atoms and to at least one substituent, which are the same or different.
